(19) United States
(12) Patent Application Publication
(10) Pub. No.: US 2009/0098066 A1
(43) Pub. Date: Apr. 16, 2009

(54) Substituted bicyclo [2.2.2] oct/5-ene compounds and their use as cooling agents

(76) Inventors: Christophe C. Galopin, Chesterfield, VA (US); Stefan Michael Furrer, Cincinnati, OH (US); Jay Patrick Slack, Loveland, OH (US); Pablo Victor Krawec, Cincinnati, OH (US); Karen Ann Bell, Loveland, OH (US)

Correspondence Address: CURATOLO SIDOTI CO., LPA, 24500 CENTER RIDGE ROAD, SUITE 280, CLEVELAND, OH, 44145, US

(21) Appl. No.: 11/990,561

(22) PCT Filed: Aug. 14, 2006
(86) PCT No.: PCT/CH2006/000428

§ 371 Date: Mar. 11, 2008

Related U.S. Application Data
(60) Provisional application No. 60/710,179, filed on Aug. 22, 2005.

(51) Int. Cl. A61K 8/36; A61K 31/045; A61K 31/19; C07C 31/135; A61K 31/215; C07C 61/12; A61K 31/195

(52) U.S. Cl. 424/48; 514/729; 514/557; 514/563; 562/400; 562/507; 568/831; 514/511; 424/49

(57) ABSTRACT

Used as cooling agents are the compounds of 1/7-isopropyl-4/5-methyl-bicyclo[2.2.2]oct-5-ene derivatives of the formula (I)

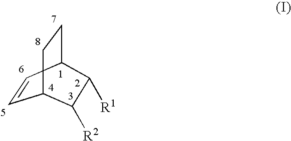

wherein
R¹ and R² are independently hydrogen, hydroxyl, hydroxymethyl, carboxy, or C(O)NHR, wherein R is methyl, ethyl, propyl, isopropyl, or cyclopropyl;
with the proviso that R¹ and R² are not both hydrogen.

The present invention relates to 1/7-isopropyl-4/5-methyl-bicyclo[2.2.2]oct-5-ene derivatives having cooling properties. The present invention refers furthermore to a process of their production and to consumer products comprising them.

In the flavor and fragrance industry there is an ongoing demand for compounds having unique cooling properties that provide the user with a pleasing cooling effect and which are suitable for use in a variety of products, particularly in ingestible and topical products.

Cooling compounds, that is, chemical compounds that impart a cooling sensation to the skin or the mucous membranes of the body, are well known to the art and are widely used in a variety of products such as foodstuffs, tobacco products, beverages, chewing gum, dentifrices, mouthwashes and toiletries.

Surprisingly it has been found that certain 1/7-isopropyl-4/5-methyl-bicyclo[2.2.2]oct-5-ene derivatives exhibit cooling properties similar to those of menthol, which is widely-used as a cooling agent. Furthermore, the provided compounds are odourless and tasteless, which makes them easier to use in food products without negatively affecting the odour- and/or taste profile of the food product to which they are added.

Accordingly, the present invention refers in one of its aspects to the use as a cooling agent of a compound of formula (I)

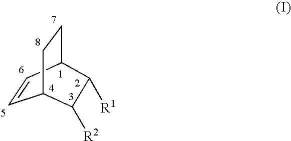

wherein
the compound of formula (I) is substituted at C-1 with isopropyl and at C-4 with methyl; or
the compound of formula (I) is substituted at C-7 with isopropyl and at C-5 with methyl; and
R¹ and R² are independently hydrogen, hydroxyl, hydroxymethyl, carboxy, or C(O)NHR,
wherein R is methyl, ethyl, propyl, isopropyl, or cyclopropyl; with the proviso that R¹ and R² are not both hydrogen.

Non-limiting examples are 1,4-substituted compounds of formula (I) wherein R¹ is hydroxyl, hydroxymethyl, carboxy, or C(O)NHR, wherein R is selected from methyl, ethyl, propyl, isopropyl, or cyclopropyl and R² is hydrogen, hydroxyl, hydroxymethyl, or C(O)NHR, wherein R is selected from methyl, ethyl, propyl, isopropyl, or cyclopropyl.

Further non-limiting example compounds may be selected from the list of 1,4-substituted compounds of formula (I) wherein R¹ is hydrogen, hydroxyl, or hydroxymethyl and R² is C(O)NHR, wherein R is selected from methyl, ethyl, propyl, isopropyl, or cyclopropyl; and compounds of formula (I) wherein R² is hydrogen, hydroxyl, or hydroxymethyl and R¹ is C(O)NHR, wherein R is selected from methyl, ethyl, propyl, isopropyl, or cyclopropyl.

In particular embodiments are compounds of formula (I) selected from the list consisting of 1-isopropyl-4-methyl-bicyclo[2.2.2]oct-5-ene-2,3-dicarbinol, N-ethyl 1-Isopropyl-4-methyl-bicyclo[2.2.2]oct-5-ene-2-carboxamide, 1-isopropyl-4-methyl-bicyclo[2.2.2]oct-5-ene-2-carboxylic acid, 1-isopropyl-4-methyl-bicyclo[2.2.2]oct-5-ene-3-carboxylic acid, 1-isopropyl-4-methyl-bicyclo[2.2.2]oct-5-ene-2-carboxylic acid propylamide and 5-methyl-7-isopropyl-bicyclo[2.2.2]oct-5-en-2-ol.

In certain embodiments the compounds as hereinabove described are in their endo-form.

The compounds of formula (I) comprise several chiral centres and as such may exist as a mixture of stereoisomers, or they may be resolved as isomerically pure forms. Resolving stereoisomers adds to the complexity of manufacture and purification of these compounds, and so the compounds may be used as mixtures of their stereoisomers simply for economic reasons. However, if it is desired to prepare individual stereoisomers, this may be achieved according to methods known in the art, e.g. preparative HPLC and GC, crystallization or stereoselective synthesis.

The compounds of formula (I) may be used in products that are applied to mucous membranes such as oral mucosa, or the skin, to give a cooling sensation. By “applying” is meant any form of bringing into contact, for example, oral ingestion or, in the case of tobacco products, inhalation. In the case of application to the skin, it may be, for example, by including the compound in a cream or salve, or in a sprayable composition. There is therefore also provided a method of providing a cooling effect to the mucous membrane or skin by applying thereto a product comprising an effective amount of a compound as hereinabove described.

Products that are applied to the oral mucosa may include foodstuffs and beverages taken into the mouth and swallowed, and products taken for reasons other than their nutritional value, e.g. tablets, mouthwash, throat sprays, dentifrices and chewing gums. Products that are applied to the skin may be selected from perfumes, toiletries, lotions, oils and ointment applicable to the skin of the human body, whether for medical or other reasons. Accordingly, in a further aspect there is provided a composition comprising an amount of at least one compound of formula (I) sufficient to stimulate the cold receptors in the areas of the skin or mucous membrane with which the composition comes into contact and thereby promote the desired cooling effect. A cooling effect may be achieved upon application of a product, for example, mouthwash or chewing gums, to the mucous membrane, e.g. oral mucosa, comprising less than 5000 ppm, in certain embodiments between 300 and 3000 ppm, such as about 1500 ppm, of a compound of formula (I). If used for beverages the addition of about 15 ppm may be sufficient to achieve a cooling effect.

Thus there is further provided an end-product selected from the group consisting of topical products, oral care products, nasal care products, toilet articles, ingestible products and chewing gum, which comprises a product base and an effective amount of at least one cooling compound of formula (I).

The compounds as hereinabove described may be used alone or in combination with other cooling compounds known in the art, e.g. menthol, menthone, isopulegol, N-ethyl p-menthanecarboxamide (WS-3), N,2,3-trimethyl-2-isopropylbutanamide (WS-23), menthyl lactate, mono-menthyl succinate (Physcool), mono-menthyl glutarate, O-menthyl glycerine (CoolAct 10) and 2-sec-butylcyclohexanone (Freskomenthe).

Whereas 1,4-substituted compounds of formula (I) wherein one of R¹ and R² is hydrogen and the other is hydroxymethyl have been described as adducts which may by obtained by Diels-Alder reaction of 1,3-p-menthadiene with acrolein by Matsubara et al.; Nippon Kagaku Zasshi (1971), 92(10), 874-876, other compounds falling within formula (I) have not been described, and are novel.

Also known from literature is 5-methyl-7-isopropyl-bicyclo[2.2.2]oct-5-en-2-ol and 5-methyl-7-isopropyl-bicyclo[2.2.2]oct-5-en-3-ol. Both compounds had been prepared for the structure determination of the corresponding ketone which was discovered in the root oil of Angelica archangeliaca L. (S. Escher et al., Helvetica Chimica Acta—Vol. 62 (7), 1979, 2061-2072).

Thus, in a further aspect there is provided a 1,4-substituted compound of formula (Ia)

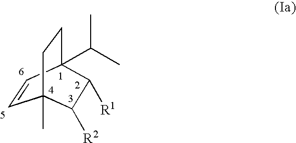

wherein R¹ and R² are independently hydrogen, hydroxyl, hydroxymethyl, or C(O)NHR, wherein R is methyl, ethyl, propyl, isopropyl, or cyclopropyl; with the proviso that
i) R¹ and R² are not both hydrogen; or
ii) if R¹ is hydrogen R² is not hydroxymethyl; or
iii) if R² is hydrogen R¹ is not hydroxymethyl.

The compounds of formula (I) may be prepared by the reaction of alpha-terpinene or alpha-phellandrene with the corresponding alkenes (acrylate/maleate) to give the corresponding Diels-Alder adduct. Depending on the alkene used, the resulting adduct is then further reduced with lithium aluminium hydride, coupled with an amine or reacted with KOH and subsequently reduced, resulting in further compounds of formula (I). The reaction may be carried out in an oxygenated solvent such as methyl-tert-butylether or tetrahydrofuran.

If the reaction is maintained at room temperature, i.e. about 20 to 25° C., the endo-adduct will be formed exclusively. If the reaction is run at higher temperature at about 50° C. to about 180° C., a mixture of endo-/exo-adducts is obtained, which may be separated by column chromatography, if it is desired. The prefixes “exo-” and “endo-” are well defined for person skilled in the art of Diels-Alder reactions.

The compositions and methods are now further described with reference to the following non-limiting examples.

### EXAMPLE 1

### 1-Isopropyl-4-methyl-bicyclo[2.2.2]oct-5-ene-2,3-dicarbinol

A) In 250 mL of MtBE, are dissolved 52 g of maleic anhydride. To the stirred solution, are added dropwise 100 mL of neat alpha-terpinene. The mixture is stirred at room temperature overnight. The solvent is then evaporated and 143 g of 1-isopropyl-4-methyl-bicyclo[2.2.2]oct-5-ene-2,3-dicarboxylic anhydride are recovered as a slightly yellow oil that solidifies over time.

B) In 100 mL of MtBE, are suspended 2.6 g of lithium aluminum hydride. To the stirred solution, 10 g of the above Diels-Alder adduct (1-isopropyl-4-methyl-bicyclo[2.2.2]oct-5-ene-2,3-dicarboxylic anhydride) in 50 mL of MtBE are added dropwise over the course of 1 hour. The mixture is then heated at reflux overnight. The solution is let cool down to room temperature and is quenched by careful addition of 25 mL of 1N aqueous NaOH. The mixture is stirred at room temperature until a white precipitate forms. The precipitate is filtered out and the solvent is evaporated to give 8 g of residue which is purified on silica gel.

### 1-Isopropyl-4-methyl-bicyclo[2.2.2]oct-5-ene-2,3-dicarboxylic anhydride

MS/EI: 234 (M^{+•}), 136, 135, 121, 119, 93, 91.

### 1-Isopropyl-4-methyl-bicyclo[2.2.2]oct-5-ene-2,3-dicarbinol

1HNMR (300 MHz, CDCl₃) 6 in ppm: 6.13 (d, 1H), 5.98 (d, 1H), 4.19 (t, 2H), 3.89 (dd, 2H), 2.88 (s, 1H), 2.84 (s, 1H), 2.65 (m, 1H), 2.44 (m, 1H), 1.93 (heptuplet, 1H), 1.41 (m, 2H), 1.23 (3, 2H), 1.16 (s, 3H), 1.05 (d, 3H), 0.88 (d, 3H).

113CNMR (75 MHz, CDCl₃) δ in ppm: 137, 136.6, 69.1, 46.0, 45.1, 43, 35.9, 33.2, 28.3, 22.7, 22.1, 18.2, 16.5.

MS/EI: 205 (M^{+•}—H₃O^{•}), 136, 121, 93, 91

### EXAMPLE 2

### 1-Isopropyl-4-methyl-bicyclo[2.2.2]oct-5-ene-2-carboxylic acid and 1-isopropyl-4-methyl-bicyclo[2.2.2]oct-5-ene-3-carboxylic acid

15 g of alpha-terpinene are dissolved in 40 mL of ethyl acrylate. A catalytic amount of aluminium trichloride is added to the mixture which is stirred at room temperature overnight. The mixture is washed with a saturated aqueous solution of sodium bicarbonate, dried over magnesium sulfate and concentrated under reduced pressure to give 23.6 g of a yellowish oil. This oil is diluted in 6 g of Aliquat 336™ (trioctylmethylammonium chloride) and 40 g of powdered potassium hydroxide is suspended in the solution. The suspension is heated at 85° C. overnight. The reaction is partitioned between 85% aqueous phosphoric acid and MTBE. The MTBE layer is dried over magnesium sulfate and concentrated to give 18 g of a yellowish oil which is crystallized in hexane to give a mixture of 1-isopropyl-4-methyl-bicyclo[2.2.2]oct-5-ene-2-carboxylic acid and 1-isopropyl-4-methyl-bicyclo[2.2.2]oct-5-ene-3-carboxylic acid. The isomers may be separated by column chromatography.

1HNMR (300 MHz, CDCl₃) mixture of regioisomers, 6 in ppm: 6.17 and 6.09 (d, 1H), 6.09 and 5.95 (d, 1H), 2.81 and 2.5 (dd, 1H), 2.09 (heptuplet, 1H), 1.8-1.65 (m, 1H), 1.5-1.3 (m, 3H), 1.3-1.1 (m, 2H), 1.21 and 1.08 (s, 3H), 0.99 and 0.93 (d, 6H).

113CNMR (75 MHz, CDCl₃) mixture of regioisomers, 6 in ppm: 181.9, 138.2 and 134.9, 136.1 and 135.6, 49.5 and 47, 43.2 and 41.6, 36.5 and 36.0, 33.9 and 33.6, 32.9 and 30.9, 28.2 and 25.

MS/EI: 208 (M^{+•}), 136, 121, 93, 91

### EXAMPLE 3

### 1-Isopropyl-4-methyl-bicyclo[2.2.2]oct-5-ene-2-carboxylic acid and 1-isopropyl-4-methyl-bicyclo[2.2.2]oct-5-ene-3-carboxylic acid

In a 5 mL-sealed tube, 1 g of acrylic acid is dissolved in 2 g of alpha-terpinene. The mixture is heated at 180° C. for 30 minutes in the microwave cavity of an Emrys Optimizer™ from Biotage, Uppsala, Sweden. The product is let cool down and washed with hexane to give 2.2 g of a colorless oil of a mixture of endo/exo- and 2/3-regioisomers.

MS/EI: 208 (M^{+•}), 136, 121, 93, 91

### EXAMPLE 4

### N-ethyl 1-Isopropyl-4-methyl-bicyclo[2.2.2]oct-5-ene-2-carboxamide

In 20 of toluene, 2 g of the mixture of 1-isopropyl-4-methyl-bicyclo[2.2.2]oct-5-ene-2-carboxylic acid and 1-isopropyl-4-methyl-bicyclo[2.2.2]oct-5-ene-3-carboxylic acid from Example 2 are dissolved. To this mixture, are added 1.1 mL of thionylchloride and the solution is heated at reflux for 2 hours. The mixture is let cool down and is added dropwise, at 0° C., to a vigorously stirred solution of 20 mL of 2N aqueous potassium hydroxide and 1.5 mL of 70% aqueous ethylamine. The mixture is stirred at 0° C. for one hour and is extracted with ether. The ether extract are washed with aqueous sodium hydroxide, aqueous hydrochloric acid and brine. The extracts are dried over magnesium sulfate, concentrated and the residue is purified by column chromatography to give 700 mg of the title compound.

1HNMR (300 MHz, CDCl₃) 8 in ppm: 6.2 (d, 1H), 6.16 (d, 1H), 5.44 (broad s., 1H), 3.25 (quintuplet, 1H), 3.18 (quintuplet, 1H), 2.69 (dd, 1H), 1.98 (heptuplet, 1H), 2.69 (dd, 1H), 1.43 (dd, 2H), 1.28-1.17 (m, 4H), 1.16 (s, 3H), 1.08 (t, 3H), 0.98 and 0.96 (d, 6H).

113CNMR (75 MHz, CDCl₃) δ in ppm: 175.9, 140.2, 135.4, 49.9, 43.2, 34.4, 34.2, 33.2, 31, 25.3, 25.1, 19, 17.1, 15.2.

MS/EI: 235 (M^{+•}), 220, 207, 192, 136, 121, 100, 99, 93, 91

### EXAMPLE 5

### 1-Isopropyl-4-methyl-bicyclo[2.2.2]oct-5-ene-2-carboxylic acid propylamide and 1-isopropyl-4-methyl-bicyclo[2.2.2]oct-5-ene-3-carboxylic acid propylamide

A mixture of endo/exo- and 2/3-regioisomers of the title compounds was obtained following the procedure according to Example 4.

MS/EI: 249 (M^{+•}), 206, 136, 121, 93, 91.

### EXAMPLE 6

### 7-isopropyl-5-methyl-bicyclo[2.2.2]oct-5-en-2-ol

A) In a 5 mL-sealed tube, 3.0 g of alpha Phellandrene and 1.93 g of chloro acrylonitrile were added and heated in the microwave for 10 min at 140° C. and 15 min at 160° C. in the microwave cavity of an Emrys Optimizer™ from Biotage, Uppsala, Sweden. The reaction mixture is let cool down and purified by column chromatography. 2.3 g of 2-chloro-5-methyl-7-isopropyl-bicyclo[2.2.2]oct-5-ene-2-carbonitrile was isolated as a orange oil.

B) In a 250 mL flask, 2.0 g of 2-chloro-5-methyl-7-isopropyl-bicyclo[2.2.2]oct-5-ene-2-carbonitrile, 20 mL of dimethylsulfoxide and 2.0 g of potassium hydroxide (86%) in 1 mL of water were added and stirred at RT for 16 h.

The mixture was extracted 2× with MTBE vs. brine. The organic layers were washed 2× with diluted brine and brine, dried over MgSO₄, concentrated and filtered with MTBE/Hex, 2:8 over a silica plug. The filtrate was concentrated and 0.56 g of a yellowish liquid were isolated.

C) In a 250 mL flask, 0.5 g of 5-methyl-7-(1-methylethyl)-bicyclo[2.2.2]oct-5-en-2-one, 50 mL of Methanol and 1 g of sodium borohydride (pellets) were added and stirred at room temperature for 16 hours. The mixture was concentrated onto silica and purified by column chromatography (MTBE:Hex, 0-20%). 0.22 g yellowish liquid were obtained.

### 2-chloro-5-methyl-7-isopropyl-bicyclo[2.2.2]oct-5-ene-2-carbonitrile

MS/EI: 223 (M+•), 208, 188, 136, 118, 93, 77, 69

### 5-methyl-7-isopropyl-bicyclo[2.2.2]oct-5-en-2-one

MS/EI: 178 (M+•), 136, 119, 109, 93, 77, 65

### 5-methyl-7-isopropyl-bicyclo[2.2.2]oct-5-en-2-ol

MS/EI: 180 (M+•), 136, 121, 93, 77, 65

### EXAMPLE 7

Table 1 shows further compounds which may also be prepared following the general procedure of examples hereinabove. Depending on the reaction temperature the pure endo-compound will be obtained or a mixture of endo- and exo-compounds which may be separated by column chromatography on silica gel to get the pure exo-compound.

TABLE 1

| No. | R² | R¹ |
|---|---|---|
| 6 | H | OH |
| 7 | | CH₂OH |
| 8 | | C(O)NHR, R = CH₃ |
| 9 | | C(O)NHR, R = C₂H₅ |
| 10 | | C(O)NHR, R = C₃H₇ |
| 11 | | C(O)NHR, R = iso-propyl |
| 12 | | C(O)NHR, R = cyclopropyl |
| 13 | OH | H |
| 14 | | OH |
| 15 | | CH₂OH |
| 16 | | C(O)NHR, R = CH₃ |
| 17 | | C(O)NHR, R = C₂H₅ |
| 18 | | C(O)NHR, R = C₃H₇ |
| 19 | | C(O)NHR, R = iso-propyl |
| 20 | | C(O)NHR, R = cyclopropyl |
| 21 | CH₂OH | H |
| 22 | | OH |
| 23 | | C(O)NHR, R = CH₃ |
| 24 | | C(O)NHR, R = iso-propyl |
| 25 | | C(O)NHR, R = cyclopropyl |
| 26 | C(O)NHR | H |
| 27 | R = CH₃ | OH |
| 28 | | CH₂OH |
| 29 | C(O)NHR | H |
| 30 | R = C₂H₅ | OH |
| 31 | | CH₂OH |
| 32 | C(O)NHR | H |
| 33 | R = iso-propyl | OH |
| 34 | | CH₂OH |
| 35 | C(O)NHR | H |
| 36 | R = cyclopropyl | OH |
| 37 | | CH₂OH |

### EXAMPLE 8

### Cooling Intensity

A small group of panelists had been asked to taste various aqueous solutions of compounds of formula (I) and indicate which solutions had a cooling intensity similar or slightly higher than that of a solution of menthol at 2 ppm. The results are shown in Table 2.

TABLE 2
Experiments on cooling intensity

| | | Con- | |
|---|---|---|---|
| Chemical | | centration | Odor |
| Comparison: I-Menthol, 2 ppm solution | | | Minty |
| N-ethyl p-menthanecarboxamide (WS-3) | | 1.5 ppm | None |
| Formula (I), | and R¹ = R² = —CH₂OH | 2 ppm | None |
| substituted at C-1 | (compound of Example 1) | | |
| with isopropyl and | and R¹ = H, R² = —C(O)OH | 2 ppm | None |
| at C-4 with methyl | (compound of Example 2) | | |
| | and R¹ = H, R² = | 3 ppm | None |
| | —C(O)NHC₂H₅ | | |
| | (compound of Example 4) | | |
| Formula (I), | and R¹ = —OH, R² = H | 5 ppm | slightly |
| substituted at C-7 | (compound of Example 6) | | woody |
| with isopropyl and | | | |
| at C-5 with methyl | | | |

### EXAMPLE 9

### Application in Chewing Gum

| | |
|---|---|
| Gum Base Flama-T* | 25.18 g |
| Compound of Example 1 | 0.10 g |
| Peppermint oil | 1.00 g |
| Corn Syrup | 17.22 g |
| Sugar | 55.17 g |
| Glycerine | 1.33 g |

*Flama-T is a trademark of Cafosa gum, Barcelona (Spain)

All the ingredients are mixed in the pre-warmed gum base. The mixture is spread in thick films, cooled down and cut in sticks. A gum stick is chewed by a panelist for 15 minutes. An immediate cooling sensation is felt in all areas of the mouth. No off-note was observed.

### EXAMPLE 9

### Application in Beverage

1.2 mL of a 0.5% ethanolic solution of 1-isopropyl-4-methyl-bicyclo[2.2.2]oct-5-ene-2,3-dicarbinol is added in a 355 mL (12 fl oz.) can of clear lemon/lime soda. A panelist experiences an immediate cooling sensation in the mouth with none of the throat burning that is characteristic of WS-3.

Although the invention has been described in detail through the above detailed description and the preceding examples, these examples are for the purpose of illustration only and it is understood that variations and modifications can be made by one skilled in the art without departing from the spirit and the scope of the invention. It should be understood that the embodiments described above are not only in the alternative, but can be combined.

1. (canceled)

2. (canceled)

3. A method of providing a cooling effect to the skin or mucosa membranes by applying thereto a compound of formula (I) 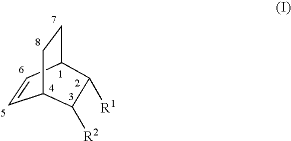
wherein
the compound of formula (I) is substituted at C-1 with isopropyl and at C-4 with methyl; or
the compound of formula (I) is substituted at C-7 with isopropyl and at C-5 with methyl; and
R¹ and R² are independently hydrogen, hydroxyl, hydroxylethyl, carboxy, or C(O)NHR, wherein R is methyl, ethyl, propyl, isopropyl, or cyclopropyl;
with the proviso that R¹ and R² are not both hydrogen.

4. (canceled)

5. A product that provides a cooling effect to the skin or mucous membranes, which product comprises at least one compound of formula (I) 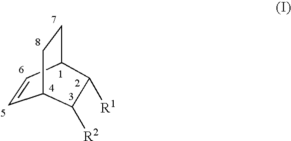
wherein
the compound of formula (I) is substituted at C-1 with isopropyl and at C-4 with methyl; or
the compound of formula (I) is substituted at C-7 with isopropyl and at C-5 with methyl; and
R¹ and R² are independently hydrogen, hydroxyl, hydroxymethyl, carboxy, or C(O)NHR, wherein R is methyl, ethyl, propyl, isopropyl, or cyclopropyl;
with the proviso that R¹ and R² are not both hydrogen.

6. A product according to claim 5 wherein the product is selected from the group consisting of topical products, oral care products, nasal care products, toilet articles, ingestible products and chewing gum, comprising a product base and an effective amount of the at least one compound of formula (I), or a mixture thereof.

7. A compound of formula (Ia) 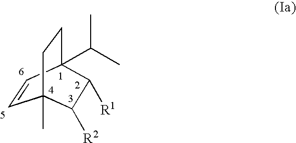
wherein
R¹ and R² are independently hydrogen, hydroxyl, hydroxymethyl, or C(O)NHR, wherein R is methyl, ethyl, propyl, isopropyl, or cyclopropyl;
with the proviso that
i) R¹ and R² are not both hydrogen; or
ii) if R¹ is hydrogen R² is not hydroxymethyl; or
iii) if R² is hydrogen R¹ is not hydroxymethyl.

8. A compound according to claim 7 selected from the list consisting of 1-isopropyl-4-methyl-bicyclo[2.2.2]oct-5-ene-2,3-dicarbinol, N-ethyl 1-Isopropyl-4-methyl-bicyclo[2.2.2]oct-5-ene-1-carboxamide, and 1-isopropyl-4-methyl-bicyclo[2.2.2]oct-5-ene-2-carboxylic acid propylamide.

9. A compound according to claim 7 wherein
R² is hydrogen and R¹ is selected from hydroxyl, C(O)NHCH₃, C(O)NHC₂H₅, C(O)NH(CH₂)₂CH₃, C(O)NH-iso-propyl, and C(O)NH-cyclopropyl; or
R² is hydroxyl and R¹ is selected from hydrogen, hydroxyl, hydroxymethyl, C(O)NHCH₃, C(O)NHC₂H₅, C(O)NH(CH₂)₂CH₃, C(O)NH-iso-propyl, and C(O)NH-cyclopropyl; or
R² is hydroxymethyl and R¹ is selected from hydroxyl, C(O)NHCH₃, C(O)NH-iso-propyl, and C(O)NH-cyclopropyl; or
R² is C(O)NHCH₃ and R¹ is selected from hydrogen, hydroxyl or hydroxymethyl; or
R² is C(O)NHC₂H₅ and R¹ is selected from hydrogen, hydroxyl or hydroxymethyl; or
R² is C(O)NH-iso-propyl and R¹ is selected from hydrogen, hydroxyl or hydroxymethyl; or
R² is C(O)NH-cyclopropyl and R¹ is selected from hydrogen, hydroxyl or hydroxymethyl.

10. The product according to claim 5 comprising 1,4-substituted compounds of formula (I) wherein R¹ is hydroxyl, hydroxymethyl, carboxy, or C(O)NHR wherein R is selected from methyl, ethyl, propyl, isopropyl, or cyclopropyl; and R² is hydrogen, hydroxyl, hydroxymethyl, or C(O)NHR wherein R is selected from methyl, ethyl, propyl, isopropyl, or cyclopropyl.

11. The product according to claim 5 comprising 1,4-substituted compounds of formula (I) wherein R¹ is hydrogen, hydroxyl, or hydroxymethyl and R² is C(O)NHR, wherein R is selected from methyl, ethyl, propyl, isopropyl, or cyclopropyl.

12. The product according to claim 5 comprising compounds of formula (I) wherein R² is hydrogen, hydroxyl, or hydroxymethyl and R¹ is C(O)NHR, wherein R is selected from methyl, ethyl, propyl, isopropyl, or cyclopropyl.

13. The product according to claim 5 wherein the compound of formula (I) is at least one compound selected from the group consisting of 1-isopropyl-4-methyl-bicyclo[2.2.2]oct-5-ene-2,3-dicarbinol, N-ethyl 1-isopropyl-4-methyl-bicyclo[2.2.2]oct-5-ene-2-carboxamide, 1-isopropyl-4-methyl-bicyclo[2.2.2]oct-5-ene-2-carboxylic acid, 1-isopropyl-4-methyl-bicyclo[2.2.2]oct-5-ene-3-carboxylic acid, 1-isopropyl-4-methyl-bicyclo[2.2.2]oct-5-ene-2-carboxylic acid propylamide, 1-isopropyl-4-methyl-bicyclo[2.2.2]oct-5-ene-3-carboxylic acid propylamide and 5-methyl-7-isopropyl-bicyclo[2.2.2]oct-5-ene-2-ol.

14. The product according to claim 5 further comprising an additional cooling compound.

15. The product according to claim 14 wherein the additional cooling compound comprises at least one of menthol, menthone, isopulegol, N-ethyl p-methanecarboxamide, N,2,3-trimethyl-2-isopropylbutanamide, menthyl lactate, mono-menthyl succinate, mono-menthyl glutarate, O-menthyl glycerine, or 2-sec-butylcyclohexanaone.

16. A method of providing a cooling effect to the skin or mucosa membranes by apply in a thereto a product according to claim 5.

17. The method according to claim 16 wherein applying to the mucosa membranes comprises oral ingestion or in the case of tobacco products, inhalation.

18. The method according to claim 16 therein the products applied to the oral mucosa comprise at least one of foodstuffs, tobacco products, beverages, tablets, mouthwashes, throat sprays, dentifrices or chewing gums.

19. The method according to claim 18 wherein the products applied to the oral mucosa comprise less than 5000 ppm of the compound of formula I; optionally between 300 and 3000 ppm of the compound of formula I; further optionally about 15 ppm of the compound of formula I when the product is a beverage.

20. The method according to claim 16 wherein the products applied to the skin comprise at least one of perfumes, toiletries, lotions, oils, ointments, creams, salves, or sprayable compositions.

21. The product according to claim 5 wherein the product comprises at least one of foodstuffs, tobacco products, beverages, tablets, mouthwashes throat sprays, dentifrices, chewing gums, perfumes, toiletries, lotions, oils, ointments, creams, salves, or sprayable compositions.
